(19) United States
(12) Patent Application Publication
(10) Pub. No.: US 2024/0320874 A1
(43) Pub. Date: Sep. 26, 2024

(54) SYSTEMS AND METHODS FOR EMULATING A CHEST X-RAY IMAGE FROM CHEST SOUNDS

(71) Applicant: Meera IYER, Fremont, CA (US)

(72) Inventor: Meera IYER, Fremont, CA (US)

(73) Assignee: Meera IYER, Fremont, CA (US)

(21) Appl. No.: 18/616,086

(22) Filed: Mar. 25, 2024

Related U.S. Application Data
(60) Provisional application No. 63/491,957, filed on Mar. 24, 2023.

(51) Int. Cl. G06T 11/00; A61B 7/00; A61B 7/04

(52) U.S. Cl. CPC G06T 11/00; A61B 7/003; A61B 7/04; G06T 2210/41

(57) ABSTRACT

Systems and methods for translating sounds captured from a patient's thoracic cavity, either by a stethoscope or other suitably sensitive sensor, and through a machine learned process translating that sound data into an emulation of an X-ray, typically a chest X-ray. The sensor or sensors can be mounted in a vest worn by the patient, who can be remotely located. The thoracic sounds are converted to an audio embedding by a spectrogram converter coupled with one or more neural networks for feature extraction and the audio embedding is then processed in an image generator neural network trained on chest X-rays to generate the emulation image.

### RELATED APPLICATION

The present application is a conversion of and claims the benefit of U.S. Patent Application Ser. 63/491,957, filed Mar. 24, 2023, entitled Systems and Methods for Generating a Chest X-Ray Image from Chest Sounds, incorporated herein by reference.

### FIELD OF THE INVENTION

The present invention relates to medical imaging and in particular to methods and systems for generating a human-interpretable X-ray-like image from sounds captured around a human body and, in an embodiment, is directed to systems and methods for generating a human-interpretable X-ray-like image of a human's thoracic cavity from sounds captured around that human's chest abdomen.

### BACKGROUND OF THE INVENTION

According to the World Health Organization, two-thirds of the world does not have access to basic radiology services such as X-rays. Even in developed countries, many patients are located sufficiently remotely from medical facilities such that they cannot readily see a physician face-to-face either because of distance, lack of transportation or lack of mobility. This is particularly true for elderly and chronically ill patients, who are among the class of patients who most frequently need medical intervention.

Imaging the body with X-rays has the additional disadvantage of exposing the body to one or more small doses of ionizing radiation. Such exposure is associated with a slightly higher risk of developing cancer later on in life. Diseases of the lungs or other parts of the thoracic cavity can represent some of the most challenging illnesses to diagnose without the patient coming to a medical facility, since chest X-ray machines and other similar diagnostic tools are typically massive devices operated in bunker-type spaces within a medical facility. Treatment of remotely located patients who present with symptoms indicating such illnesses are among those who most need access to medical facilities but cannot get it. Thus, there has been a long-felt need for systems and methods that aid in the remote diagnosis of illnesses involving the thoracic cavity and particularly the lungs.

Today, many machine learning-based methods are able to diagnose certain heart and lung diseases from stethoscope sounds or from chest X ray images. While such information provides numerous benefits, what has been missing is any way to translate sounds from the thoracic cavity into images of that cavity similar in quality to a chest X-ray. Thus there has been a long felt need for systems and methods that can convert sounds captured from the thoracic cavity into emulations of chest X-rays of sufficient quality that they provide actionable data to an operator.

### SUMMARY OF THE INVENTION

In an embodiment, the present invention converts sounds captured from a patient's thoracic cavity, either by a stethoscope or other suitably sensitive sensor, and through a machine learned process translates the sound data into an emulation of an X-ray, typically a chest X-ray. The sensor can be a plurality of sensors rather than just a stethoscope, for example a vest capable of being worn by a remotely-located patient. In some embodiments, the vest can have a plurality of sensors positioned, for example, in various locations where, when the vest is worn, those sensors are positioned against the patient similar to the locations where a physician would place a stethoscope if the patient was being seen in person.

The captured audio signals from the thoracic cavity are encoded into an audio embedding of a predetermined size appropriate for the scale of the final image. The embeddings are then provided to an image generator which provides as its output an image which emulates a conventional X-ray. The encoding process involves a plurality of preprocessing steps, including performing a spectrogram conversion using methods known to those skilled in the art. In an embodiment the spectrogram conversion involves a Fast Fourier Transform to convert the time-based segments to the frequency spectrum, and then converting those values to power spectrum values by squaring the coefficients, in a manner known in the art, to yield a set of FFT magnitudes representative of each time window and frequency. An optional filtering step, for example a window function, can be implemented prior to the FFT transform to minimize edge effects and improve frequency response.

In such an embodiment, the FFT magnitudes are fed to a convolutional neural network (CNN) to perform feature extraction and then to a recursive neural network (RNN) to complete the encoding that results in the audio embedding. In embodiments with multiple audio sensors, each sensor's audio output comprises a separate channel and undergoes spectrogram conversion. The resulting FFT magnitudes are all then provided to the CNN and the RNN to create the audio encoding process and provide the resulting audio embedding representative of the multiple audio input channels. In at least some embodiments, the captured multichannel sound data can result in more accurate emulation of a chest X-ray image. Further, while the invention works well for audio in the human hearing range, sensors that respond to the infrasonic and ultrasonic bands of the sound spectrum, either alone or in combination with other frequency ranges, can further enhance the accuracy of the emulated chest X-ray image.

It is therefore one object of the present invention to provide a system and method by which thoracic sounds can be translated to emulate a chest X-ray.

It is another object of the present invention to provide a system and method for remotely monitoring a patient's thoracic cavity by capturing thoracic sounds.

A still further object of the present invention is to provide a wearable multisensor system capable of providing to a host one or more audio signals for processing into an emulation of a chest X-ray.

These and other objects of the present invention can be better appreciated from the following detailed description of the invention taken together with the appended Figures.

### FIGURES

FIG. 1 illustrates in functional block diagram form an embodiment of an X-ray emulation system in accordance with the invention.

FIG. 2A illustrates in flow diagram form an embodiment of a single channel audio encoder in accordance with the present invention.

FIG. 2B illustrates in flow diagram an embodiment of a multi-channel audio encoder in accordance with the present invention.

FIG. 3 illustrates in flow diagram form an embodiment of a process for training the audio encoder in accordance with the present invention.

FIG. 4 illustrates in flow diagram an embodiment of a process for training the image generator in accordance with the present invention.

FIG. 5 illustrates an embodiment of a wearable vest having a plurality of audio sensors suitable for providing a multi-channel input to the audio encoder in accordance with the present invention.

FIG. 6 illustrates an embodiment of a hardware platform configured to execute the software functions described herein.

### DETAILED DESCRIPTION OF THE INVENTION

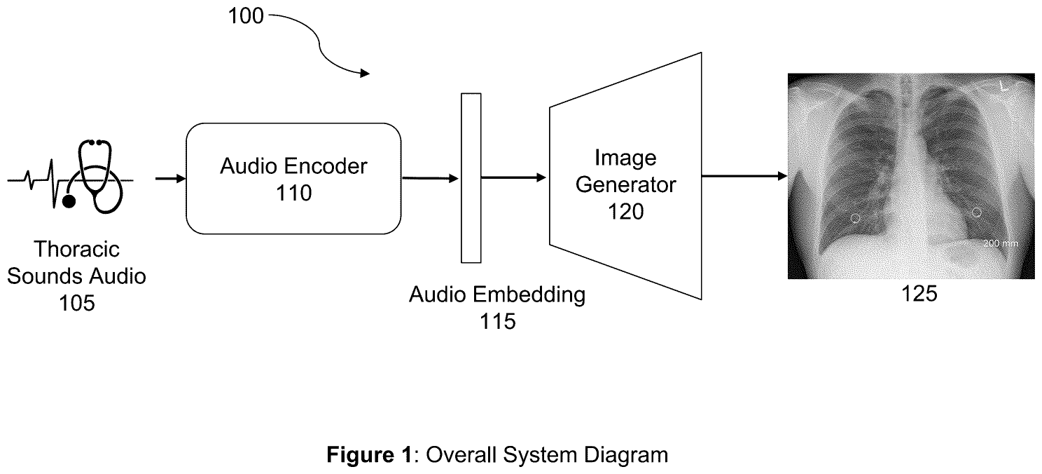

Referring to FIG. 1, shown therein at a high level is an embodiment of a method 100 for translating thoracic sounds recorded from a patient into an image that emulates a chest or similar X-ray of that patient. The thoracic sound data is encoded into an audio embedding of a certain size, for example a 1024-element vector. It will be appreciated that the vector size can be increased or decreased depending up the desired scale of the output image produced by the invention, for example vector sizes of 2048 or 4096 elements. A 1024-element vector can be used to generate a 256×256 pixel image or large, with larger vectors appropriate for larger final image sizes. Those skilled in the art will appreciate that the vector which comprises the embedding is a set of real numbers that are compressed representations of the image data. The embedding is passed through an image generator that synthesize an image substantially identical to the image that would result from a chest X-ray of that patient.

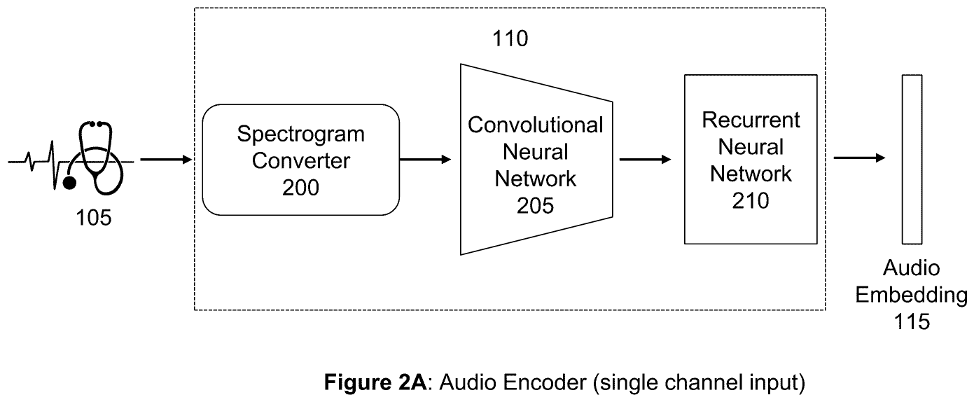

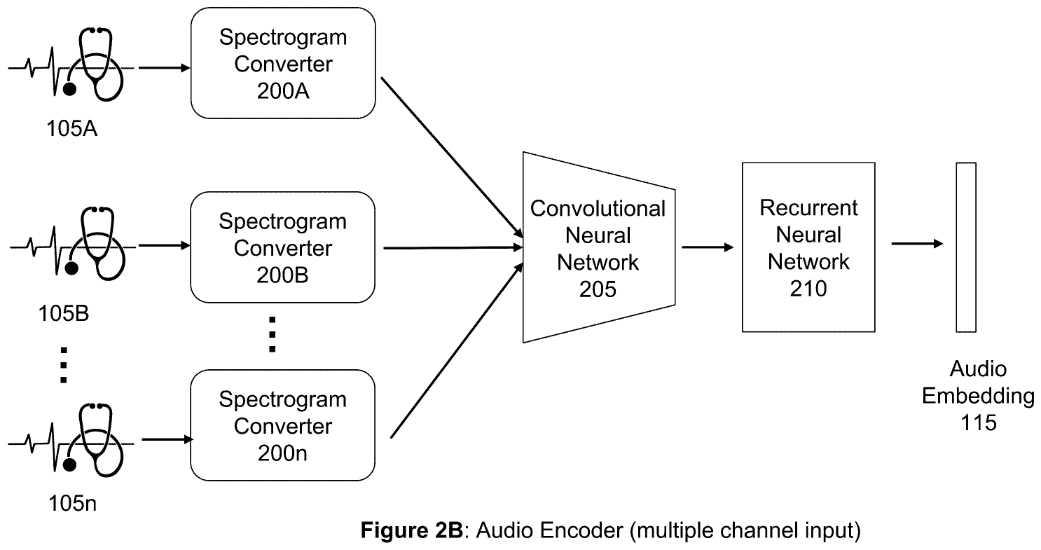

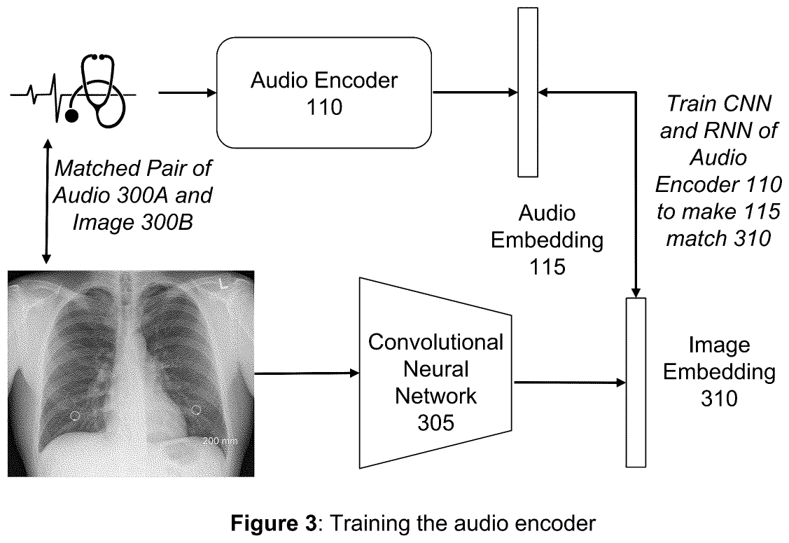

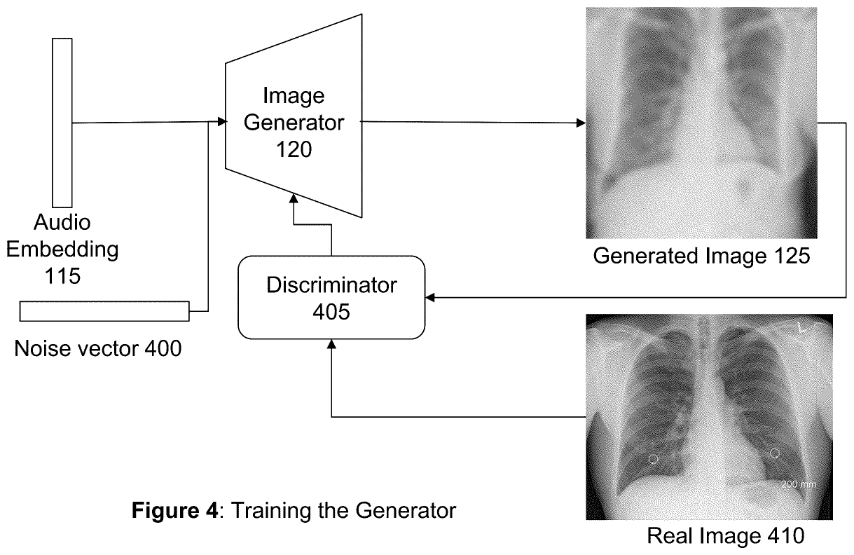

Thus, in FIG. 1, one or more channels of thoracic sounds 105 are provided to an audio embedding preprocessing step 110, explained in greater detail in connection with FIGS. 2A and 2B, below. The output of the preprocessing step 110 results in an audio embedding shown at 115. That embedding is then processed by an image generator 120 which translates the embedding into an image 125 as explained in greater detail hereinafter, including the discussion of FIGS. 3 and 4.

Referring to FIG. 2A, the audio encoder 110 can be understood in greater detail. The thoracic sound data 105 is first converted into a spectrogram image in a manner known in the art, indicated in FIG. 2 at 200. This is done as follows: The audio signal 105 is divided into short segments or frames. Optionally, each segment s(t) is multiplied by a window function w(t) (such as a Hamming or Hann window although other window functions will also work) to minimize edge effects and improve the frequency resolution. The window function w(t) is chosen such that the central part of the signal s(t) is emphasized. Common choices for the window function are the Hamming or Hann functions. Next, for each segment, a Fast Fourier Transform (FFT) is performed which converts the time domain signal within each window into the frequency domain. The square of the magnitude of the Fourier Transform coefficients is then calculated, which converts the complex Fourier coefficients into a power spectrum and shows the power present at each frequency component within the segment. Optionally, if it is desirable to display this result for viewing, the power spectrum values for each segment are then mapped to any convenient scale, for example colors using a color mapping function. One choice for the color mapping function is grayscale where low magnitudes are represented as darker shades of gray and large values for whiter shades. Another choice may be heatmap based coloring where low magnitudes are mapped to blue, moderate values to yellow, and large values to red. The colored segments are laid out in sequence along the time axis to produce the spectrogram image.

In at least some embodiments, the thoracic sound data can comprise frequencies audible to the human ear. In other embodiments, the thoracic sound data can include ultrasonic frequencies either alone or in combination with human audible frequencies. In still other embodiments, the spectrogram image can comprise a Mel spectrogram image. In further embodiments, the thoracic sound data may include, in addition to human audible frequencies, infrasonic frequencies.

In an embodiment, the spectrogram image, specifically the power spectrum values resulting from the squares of the FFT coefficients, is passed through one or more neural networks in order to produce a final audio embedding as described below. In the spectrogram image, each column corresponds to a time ordered set of Fourier magnitudes for a specific frequency. Each row represents all Fourier spectrum magnitudes for a given time window. The spectrogram image is passed through an image feature extractor such convolutional neural network (CNN) 205 and then through a recurrent neural network (RNN) 210 to create the audio embedding 115. In an embodiment, the CNN 205 can include several successive layers of a group of convolutional and batch normalization operations followed by a Rectified Linear Unit (ReLU) activation function. Optionally, a MaxPool operation may be included in between layers. In an embodiment, the group of layers can be designed so that the final resulting frequency dimension is one, with the temporal dimension having more than one value—in other words, a column vector such as 32×1 where 32 corresponds to the temporal dimension and 1 corresponds to the frequency dimension. Depending upon the embodiment and the specific implementation, there may be several channels of such a vector, for example 1024. As the data is a temporal sequence, inputting this data to a Long Short Term Memory (LSTM) network, can help capture temporal dependencies. As is well known in the art, LSTMs can be configured to have any number of hidden layers, such as 1024, and to produce a final output vector of a chosen dimension, such as 1024 or other convenient size suited to the scale of the image. As is also well-known, the LSTM can optionally also be configured to be bidirectional where information can flow not only from earlier to later times, but also from later times to earlier times.

Referring next to FIG. 2B, in another embodiment, there may be multiple channels of thoracic sound data 105A-105n, captured from a plurality of locations on the subject's thorax. A spectrogram is generated for each channel's sound data and input into the neural networks, e.g. CNN 205 and RNN 210, as multiple layers of input data. As is well known, several independent channels of spectrogram images, such as those from 105A-105n taken from different locations on the thorax, can be analyzed simultaneously by the neural network architecture described above, similar to the way color images are input as three independent channels of red, green, and blue images into a CNN for the task of image classification. Thus, the combination of the CNN 205 and RNN 210 operates on the multichannel input from spectrum converters 200A-200n to yield an audio embedding 115 that captures the information for all of the input channels.

Referring to FIG. 3, in a particular embodiment the audio encoder 110 may be trained on data comprising matched pairs of thoracic sound data together with a chest X-ray from the same patient taken at roughly the same time, indicated in FIG. 3 at 300A and 300B respectively. To train the audio encoder 110, the chest X-ray image is first passed through a pre-trained image encoder 305 to generate an image embedding. The image encoder 305 can be, for example, a CNN that is trained on a standard dataset. For example, the CNN 305 may be first trained an a chest X-ray image dataset to perform a task such as disease classification. Such a network may be trained, for example, on data from the NIH chest X-Ray dataset, or the CheXpert dataset from Stanford University. As is well known in the art, the penultimate layer of the neural network provides a good representation of the chest X-Ray image. Software frameworks such as TorchXRayVision that provide CNNs specifically pretrained on chest X-Ray images can also be used. In general, the CNN 305 can be any network that has been trained to process chest X-Ray images whose penultimate layer values provide the image embedding. The resulting image embedding is shown at 310.

Because the objective of the audio encoder training process of FIG. 3 is to cause the audio encoder 110 to yield an audio embedding 115 that matches the image embedding 315, it will be appreciated that the size of the vector generated by the pre-trained image encoder 305 will in most cases match size of the vector which forms the audio embedding 115. The audio encoder's parameters are optimized to produce approximately the same embedding as the image embedding. This paradigm of training is also known as teacher-student training.

For training the audio encoder, in one embodiment, the loss functions can be described as follows:

$$L = {L}_{JEL} + {λ}_{norm} {L}_{{norm}_{i}} + {λ}_{KDL} {L}_{KDL}$$

$$where :$$

$${L}_{JEL} = α {E}_{{y}_{j} \neq {y}_{i}} [ max ( 0 , {f}_{{s}_{i}}^{T} {f}_{{v}_{j}} - {f}_{{s}_{i}}^{T} {f}_{{v}_{i}} + {m}_{diff} ) ] + β {E}_{{y}_{j} = {y}_{i}} [ max ( 0 , {f}_{{s}_{i}}^{T} {f}_{{v}_{j}} - {f}_{{s}_{i}}^{T} {f}_{{v}_{i}} + {m}_{same} ) {L}_{{norm}_{i}} = {\sum }_{k} \vert {f}_{sik} - {f}_{vik} \vert$$

$${L}_{KDL} = KL ( softmax ( {f}_{{s}_{i}} ) \vert softmax ( {f}_{{v}_{i}} ) )$$

Losses are calculated for each triplet (vᵢ,sᵢ,yᵢ) in a batch of matched audio and image pairs for sample i, vᵢ is the image, sᵢ is the spectrogram, and yᵢ is the class label associated with the sample (for example a pathology such as “pneumonia”). fₛ_{ᵢ} is the embedding produced by the audio encoder, fᵥ_{ᵢ} is the embedding produced by the pretrained image encoder. L_{JEL} is referred to as the “Joint Embedding Loss” where available class labels for a pair of audio and image data, for example a pathology such as pneumonia, can be used to decide the significance of the loss. In the equation for L_{JEL}, α and β are weights assigned to pairs that come from different and same labels respectively. E stands for the expectation, m_{diff} and mₛₐₘₑ are constants associated for different and same labels respectively. Lₙₒᵣₘ_{ᵢ} stands for the 1-norm of the feature vector for sample i. KL stands for the well-known KL divergence function that calculates a distance between two different probability distributions. softmax is the well-known function e^{x}^{ᵢ}/Σⱼe^{x}^{ⱼ} that converts a vector of numbers x into a probability distribution. Additional loss terms may accelerate training in some instances. Such additional loss terms are known in the art.

Referring to FIG. 4, in a particular embodiment, the image generator 120 may be a stacked conditional generative adversarial network, known as StackGAN v2. In this context, “conditional” means that the generation of the image is conditioned on a class label. The generator 120 is trained to generate an image conditioned on the audio embedding and a noise vector z, indicated at 400, that varies the background of the generated image. A discriminator 405 is trained simultaneously, as is known in the art, to discriminate between the generated image 125 and a real image 410. The discriminator 405 takes real images 410 and the audio embeddings 115 as positive sample pairs, whereas for negative samples the pair is the generated image 125 and the audio embedding 115. The information is input back to the generator 120 to improve the quality of the images generated. After training, the generator 120 is able to generate substantially realistic images that emulate a chest or similar X-ray of the patient who provided the associated thoracic sounds.

In an embodiment where the generator is a StackGAN v2, where images at three scales, for example 64×64, 128×128, and 256×256, are generated, the training loss functions may be as described in detail below. The discriminator 405 includes three terms: a conditional term, an unconditional term and a wrong pair term. Given a triplet (cᵢ, vᵢ, vᵢ^{w}) where, for each sample i, cᵢ denotes the class of the sample, vᵢ is the image embedding, and vᵢ^{w} is the image embedding from a class different from cᵢ, referred to here as “wrong” class. The discriminator network in the GAN is trained to minimize the loss L_{D} as defined below:

$${L}_{D} = {\sum }_{s = 1}^{3} ( {L}_{{D}_{s}}^{cond} + {L}_{{D}_{s}}^{uncond} + {L}_{{D}_{s}}^{wrong} )$$

$$Where :$$

$${L}_{{D}_{s}}^{cond} = {E}_{( {v}_{i} , {c}_{i} ) ∼ p} [ {D}_{s} ( {v}_{i} , {c}_{i} ) + ( 1 - {D}_{S} ( G ( z , {c}_{i} ) , {c}_{i} ) ) ]$$

$${L}_{{D}_{s}}^{uncond} = {E}_{( {v}_{i} , {c}_{i} ) ∼ p} [ {D}_{s} ( {v}_{i} ) + ( 1 - {D}_{S} ( G ( z , {c}_{i} ) ) ) ]$$

$${L}_{{D}_{s}}^{wrong} = {E}_{( {v}_{i} , {c}_{i} ) ∼ p} [ 1 - {D}_{S} ( {v}_{i}^{w} , {c}_{i} ) ]$$

where p is the data distribution for the pair (vᵢ, cᵢ), L_{D} is summed over three scales of images. s denotes scale. Dₛ denotes the discriminator associated with scale s and G(z, cᵢ) denotes the generator's output image given a noise vector z and class cᵢ. The generator is trained to minimize the loss L_{G} as defined below:

$${L}_{G} = {\sum }_{s = 1}^{3} {E}_{( {v}_{i} , {c}_{i} ) ∼ p} + [ {D}_{s} ( G ( z , {c}_{i} ) + {D}_{S} ( G ( z , {c}_{i} ) , {c}_{i} ) ) ]$$

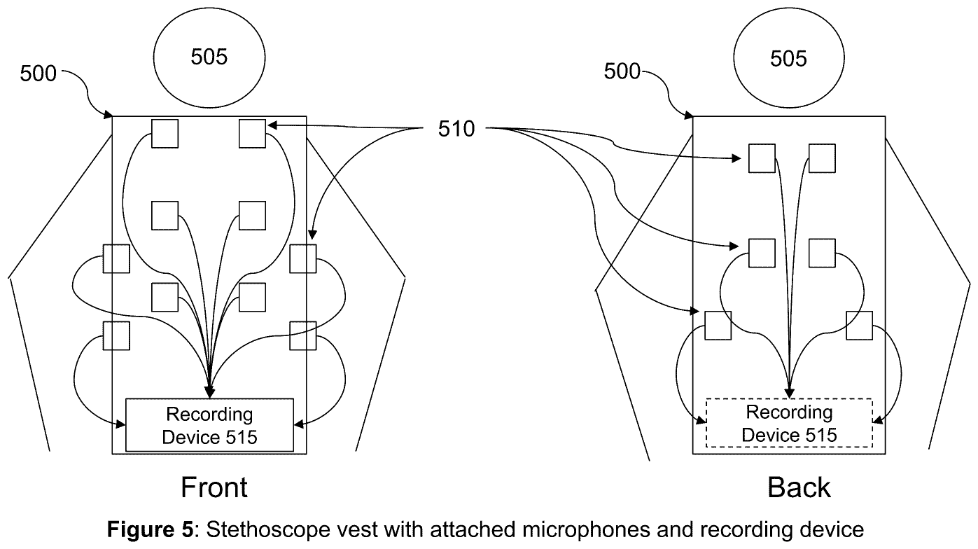

Referring to FIG. 5, a stethoscope vest 500 may be used as the source for thoracic sound data. The vest can be worn by a patient 505 in a remote location, and is typically worn sufficiently tightly against the torso that good contact is made between microphones or similar sensors 510 and the patient 505. In one embodiment, there may be, for example, sixteen sensors 510 incorporated into the vest 500 across the front and back, as depicted in FIG. 5. The exact number can vary, either fewer or more, based on the needs for a particular embodiment. For convenience, especially to avoid confusion with overlapping lines, only some of sensors 510 are indicated in FIG. 5. In an embodiment, the microphones 510 can be electret condenser microphones with a built-in amplifier although any suitably sensitive electroacoustic transducer will do in at least some embodiments. The transducers may also be filtered and/or provide external noise cancellation in some embodiments. In an embodiment, the signals from all the microphones are sent to recording device 515 which can be integrated into the vest 500 or can be external to it, or the two can be wirelessly connected via Bluetooth or similar protocol. The recording device 515 can, in some embodiments, be a processing board such as an Arduino or EPS32, which in some implementations can be configured to record all the signals simultaneously. The synchronized audio signals can be sent to a server wirelessly where the audio encoder 110 and the image generator 120 can be run in sequence, in order to produce an emulated chest X ray image.

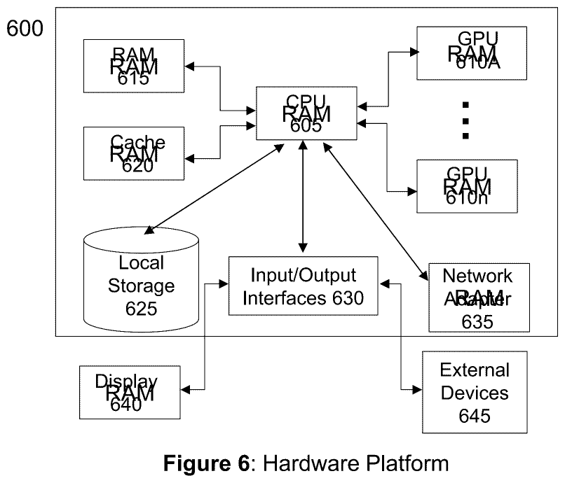

Referring next to FIG. 6, an embodiment of a hardware platform 600 suitable for executing each of the functions described herein can be appreciated. A CPU 605 communicates bidirectionally with one or more optional GPU's 610A-610n as well as RAM 615, cache 620 and local storage 625. The CPU also communicates bidirectionally with I/O interfaces 630 and network adapter 635. The I/O interfaces in turn communicate bidirectionally with a display 640 and other external devices 645 such as keyboard, mouse, and so on.

In some embodiments described herein, plural instances may implement components, operations, or structures described as a single instance and vice versa. Likewise, individual operations of one or more embodiments may be illustrated and described collectively where, alternatively, one or more of the individual operations may be performed concurrently, and the operations may be performed in an order different than that illustrated. Structures and functionalities presented as separate components in example configurations may be implemented as a combined structure or single component. Similarly, structures and functionalities presented as single components or structures may be implemented as a one or more structures or components. These and other variations, modifications, additions, and improvements fall within the scope of the subject matter herein.

Embodiments described herein as including components, modules, mechanisms, functionalities, steps, operations, or logic may comprise either software modules (e.g., code embodied on a machine-readable medium or in a transmission signal) or hardware modules. A hardware module comprises a tangible unit configured or arranged to perform the requisite operations. In example embodiments, one or more computer systems (e.g., a standalone, client or server computer system, co-located or remote from one another) or one or more hardware modules of a computer system (e.g., a processor or a group of processors) may be configured either by software (e.g., an application or application portion) or as a hardware module that operates to perform certain steps or operations as described herein.

In various embodiments, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within one or more general-purpose processors or other programmable processors) that is temporarily configured by software to perform certain operations. It will be appreciated that the implementation of a hardware module in a particular configuration may be driven by cost and time considerations.

Embodiments in which one or more hardware modules are temporarily configured (e.g., programmed), each of the hardware modules need not be configured or instantiated at any one instance in time. For example, where the hardware modules comprise a general-purpose processor and/or a graphics processor configured using software, one or more such processors may be configured as respective different hardware modules at different times. Software may accordingly configure a processor, for example, to constitute a particular hardware module at one instance of time and to constitute a different hardware module at a different instance of time.

The one or more processors may also operate to support performance of the relevant operations in a “cloud computing” environment or as a “software as a service” (SaaS). For example, at least some of the operations may be performed by a group of computers (as examples of machines including processors), these operations being accessible via a network (e.g., the Internet) and via one or more appropriate interfaces (e.g., application program interfaces (APIs).) The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Some portions of this specification are presented in terms of equations, algorithms or symbolic representations of operations on data stored as bits or binary digital signals within a machine memory (e.g., a computer memory). These equations, algorithms or symbolic representations are examples of techniques used by those of ordinary skill in the machine learning and data processing arts to convey the substance of their work to others skilled in the art. As used herein, an “algorithm” is a self-consistent sequence of operations or similar processing leading to a desired result. In this context, algorithms and operations involve manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are to be understood merely as convenient labels associated with appropriate physical quantities.

Unless specifically stated otherwise, terms such as “processing,” “computing,” “calculating,” “determining,” “presenting,” “displaying,” “generating”, “emulating” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The phrase “in an embodiment” used in various places in the specification do not necessarily all refer to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Having fully described a preferred embodiment of the invention and various alternatives, those skilled in the art will recognize, given the teachings herein, that numerous further alternatives and equivalents exist which do not depart from the invention. Thus, while particular embodiments and implementations have been illustrated and described, it is to be understood that the invention is not limited to the precise embodiments, structures and configurations disclosed herein but is to be limited only by the appended claims.

I claim:

1. A method for translating thoracic sounds of a human to an image which emulates a chest X-ray of that human comprising the steps of receiving from one or more inputs thoracic sounds of a human,
automatically performing in a computer a spectrogram conversion on the thoracic sounds from each of the one or more inputs, wherein the thoracic sounds are converted to a plurality of power spectrum values,
automatically encoding, by means of a neural network operating in the computer, the power spectrum values into an audio embedding, the neural network having been trained to match thoracic sounds to chest X-ray images, and
generating, in an image generator trained to translate thoracic sounds into images that emulate chest X-rays operating in the computer and in response to the audio embedding, an emulation of a chest X-ray of the human.

2. A system for translating thoracic sounds to an image which emulates a chest X-ray of a patient comprising
one or more inputs for receiving one or more channels of thoracic sounds,
a processor responsive to each of the one or more channels of thoracic sounds for
converting the received thoracic sounds to a plurality of power spectrum values,
generating an audio embedding representative of the power spectrum values wherein the audio embedding results from an audio encoder neural network trained to convert thoracic sounds to chest X-ray images, and
processing the audio embedding in an image generator neural network, the image generator neural network trained on chest X-rays and configured to generate an image which emulates a chest X-ray of the patient, and
outputting the image for evaluation by an operator.
